(19) United States
(12) Patent Application Publication
(10) Pub. No.: US 2018/0008362 A1
(43) Pub. Date: Jan. 11, 2018

(54) Secure Insertion Of An Insertion Device

(71) Applicants: Alexander Mellon Eaton, Fort Myers, FL (US); Gabriel Mikal Gordon, Santa Barbara, CA (US); Dyson William Hickingbotham, Wake Forest, NC (US)

(72) Inventors: Alexander Mellon Eaton, Fort Myers, FL (US); Gabriel Mikal Gordon, Santa Barbara, CA (US); Dyson William Hickingbotham, Wake Forest, NC (US)

(21) Appl. No.: 15/712,134

(22) Filed: Sep. 21, 2017

Related U.S. Application Data
(63) Continuation-in-part of application No. 14/410,567, filed on Dec. 22, 2014, now Pat. No. 9,795,767.
(60) Provisional application No. 61/692,620, filed on Aug. 23, 2012.

(51) Int. Cl. A61M 25/10; A61B 17/12; A61F 2/95; A61M 25/00; A61M 5/00

(52) U.S. Cl. CPC A61B 50/30; A61M 25/10; A61M 25/002; A61M 5/002; A61F 2/95; A61B 17/12136; A61B 2050/3013; A61B 2050/0065

(57) ABSTRACT

An insertion system and a method for protecting an insertion device, for example, a needle, a catheter, a stent, etc., from infection and/or damage before, and/or during, and/or after insertion of the insertion device into an insertion site, are provided. The insertion system includes a hub or an introducer sheath with a hub, the insertion device, and an insertion device protection apparatus. The insertion device protection apparatus includes at least two elongate protective covers detachably engaged to each other and defining a channel extending through a length of the insertion device protection apparatus for enclosing the insertion device therewithin before, and/or during, and/or after inserting the insertion device into the insertion site. The elongate protective covers are progressively disengaged from each other from a proximal end and/or a distal end of the insertion device protection apparatus before, and/or during, and/or after insertion of the insertion device into the insertion site.

### CROSS-REFERENCE TO RELATED APPLICATIONS

This application is a continuation-in-part application of non-provisional patent application titled “Systems And Methods For Performing An Injection”, application Ser. No. 14/410,567, filed in the United States Patent and Trademark Office on Dec. 22, 2014, which is a national stage application of PCT international application titled “Systems And Methods For Performing An Injection”, international application number PCT/US2013/056303, filed in the United States Patent and Trademark Office as the Receiving Office on Aug. 22, 2013, which claims priority to and the benefit of provisional patent application titled “Systems And Methods For Performing An Injection”, application Ser. No. 61/692,620, filed in the United States Patent and Trademark Office on Aug. 23, 2012. The specifications of the above referenced patent applications are incorporated herein by reference in their entirety.

### TECHNICAL FIELD

The apparatus, system, and methods disclosed herein generally relate to the field of injections. In particular, the apparatus, system, and methods disclosed herein relate to protection and injection devices, systems, and methods for preventing or substantially reducing an occurrence of an infection and/or damage to an injection device before, and/or during, and/or after an injection.

### BACKGROUND

Positioning or insertion of insertion devices, for example, needles, medical balloons, stents, etc., for injection, or catheters for prolonged infusion of solutions into a patient, inherently carries a risk of infection and/or damage to the insertion devices. There are many steps healthcare providers can take to mitigate infection, for example, by sterilization of an insertion site, that is, an injection site or an infusion site, sterilization of the insertion devices and instruments, and sterilization of the healthcare providers themselves. Damage to an insertion device is typically mitigated through careful handling of the insertion device and protective tubes and/or covers that are removed prior to use. Once an insertion device, for example, a needle or a catheter is removed from a cover that is used to protect the insertion device during shipping and storage, the insertion device is susceptible to damage and/or contamination. After removal of an insertion device from its packaging for use, conventional protective covers or sleeves seek to protect healthcare providers from accidental needle stick injuries. These protective covers are not typically designed to prevent infection and/or damage and/or contamination to the insertion device. The protective covers that initially extend over the tip of an insertion device before insertion into an insertion site are often configured to return to that position to prevent needle sticks after the insertion device is contaminated. These types of protective covers typically require a large additional force to puncture an insertion site, which is undesirable when dealing with sensitive areas of the human body or with sensitive patients. Moreover, these protective covers are not practical in applications with catheters that are to be left in place or with long devices that are to be inserted through long distances inside the human body, for example, for an angioplasty or a stent placement.

Hence, there is a long felt need for a method and a system comprising an insertion device protection apparatus for allowing secure insertion of an insertion device of any type into an insertion site, while protecting the insertion device from infection and/or damage and/or contamination before, and/or during, and/or after insertion of the insertion device into the insertion site, regardless of length of the insertion device and/or duration of the insertion of the insertion device into the insertion site. Furthermore, there is a need for an insertion device protection apparatus that can be easily removed from around the insertion device after the insertion device is inserted into the insertion site.

### SUMMARY OF THE INVENTION

This summary is provided to introduce a selection of concepts in a simplified form that are further disclosed in the detailed description of the invention. This summary is not intended to determine the scope of the claimed subject matter.

The method and the insertion system comprising the insertion device protection apparatus disclosed herein address the above recited need for allowing secure insertion of an insertion device of any type into an insertion site, while protecting the insertion device from infection and/or damage and/or contamination before, and/or during, and/or after insertion of the insertion device into the insertion site, regardless of length of the insertion device and/or duration of the insertion of the insertion device into the insertion site. Furthermore, the insertion device protection apparatus disclosed herein can be easily removed from around the insertion device after the insertion device is inserted into the insertion site. The insertion device protection apparatus disclosed herein is practical in applications with catheters that are to be left in place or with long devices that are to be inserted through long distances inside the human body, for example, for an angioplasty or a stent placement.

In an embodiment, the insertion system disclosed herein is used performing an injection at an insertion site, for example, an injection site or an infusion site. The insertion system comprises an injection device having an injection needle that penetrates the insertion site and a sleeve that houses the injection needle. The sleeve of the insertion system disclosed herein encloses the injection needle at least partially before and during the injection without penetrating the insertion site.

In an embodiment, the insertion system disclosed herein comprises an insertion assembly and an insertion device protection apparatus. In an embodiment, the insertion assembly comprises a hub and an insertion device, for example, a needle, a catheter, a medical balloon, a stent, a balloon catheter, a drug delivery device, a fluid delivery line, etc., or any combination thereof. The insertion device protection apparatus disclosed herein comprises two or more interacting parts that engage to form a channel to enclose the insertion device. The interacting parts of the insertion device protection apparatus are detachably engaged to each other and are positioned to enclose the insertion assembly. The insertion device protection apparatus provides a protective covering and protects the insertion device from infection and/or damage and/or contamination before, and/or during, and/or after insertion of the insertion device into an insertion site. The two interacting parts of the insertion device protection apparatus comprise a first elongate protective cover and a second elongate protective cover that create a channel between the two interacting parts. The channel created between the first elongate protective cover and the second elongate protective cover of the insertion device protection apparatus extends through a length of the insertion device protection apparatus and encloses the insertion device therewithin before, and/or during, and/or after insertion of the insertion device into the insertion site.

In an embodiment, the effective diameter of the channel defined between the two interacting parts of the insertion device protection apparatus is less than the diameter of the hub of the insertion assembly. In an embodiment, the insertion device protection apparatus further comprises one or more of the following structures. The first elongate protective cover of the insertion device protection apparatus comprises two or more grooves and the second elongate protective cover of the insertion device protection apparatus comprises two or more protrusions that fit into the grooves of the first elongate protective cover. The first elongate protective cover and the second elongate protective cover enclose the insertion device within the channel before, and/or during, and/or after the insertion of the insertion device into the insertion site. The first elongate protective cover is progressively disengaged from the second elongate protective cover from a proximal end and/or a distal end of the insertion device protection apparatus before, and/or during, and/or after the insertion of the insertion device into the insertion site. The progressive disengagement of the first elongate protective cover from the second elongate protective cover of the insertion device protection apparatus allows the insertion device to advance towards and be securely inserted into the insertion site.

Disclosed herein is a method for securely inserting an insertion device into an insertion site, while protecting the insertion device from infection and/or damage before and during the insertion of the insertion device into the insertion site. The method disclosed herein comprises providing an insertion system comprising the insertion assembly and the insertion device protection apparatus disclosed above; positioning the hub and the insertion device of the insertion assembly within the channel defined between the first elongate protective cover and the second elongate protective cover of the insertion device protection apparatus, where a tip of the insertion device is enclosed within the defined channel; positioning the insertion device protection apparatus with the positioned hub and the positioned insertion device of the insertion assembly proximal to the insertion site; advancing the insertion assembly enclosed in the insertion device protection apparatus towards the insertion site, while the hub of the advancing insertion assembly progressively disengages the first elongate protective cover from the second elongate protective cover from a proximal end of the insertion device protection apparatus; and inserting the insertion device of the advanced insertion assembly into the insertion site. In the method disclosed herein, the two or more interacting parts of the insertion device protection apparatus are separated as the hub of the insertion assembly advances through the insertion device protection apparatus, and the insertion device protection apparatus is removed after the insertion device is completely inserted into the insertion site.

Disclosed herein is an embodiment of the method for securely inserting an insertion device into an insertion site, while protecting the insertion device from infection and/or damage before and during the insertion of the insertion device into the insertion site. In this embodiment, the method comprises providing an insertion system comprising an introducer sheath with a hub, an insertion device, and the insertion device protection apparatus disclosed above; inserting a distal end of the introducer sheath into the insertion site; enclosing the insertion device within the channel defined between the first elongate protective cover and the second elongate protective cover of the insertion device protection apparatus; positioning the insertion device protection apparatus with the enclosed insertion device proximal to the hub of the introducer sheath; advancing the insertion device enclosed in the insertion device protection apparatus towards the hub and allowing the hub to progressively disengage the first elongate protective cover from the second elongate protective cover from the distal end of the insertion device protection apparatus, while the insertion device enters the introducer sheath via the hub and advances towards a treatment site distal to the insertion site; and inserting the insertion device free of the first elongate protective cover and the second elongate protective cover of the insertion device protection apparatus into the insertion site through the distal end of the introducer sheath.

Disclosed herein is another embodiment of the method for securely inserting an insertion device into an insertion site, while protecting the insertion device from infection and/or damage before, during, and after the insertion of the insertion device into the insertion site. In this embodiment, the method comprises providing an insertion system comprising an introducer sheath with a hub, an insertion device, and the insertion device protection apparatus disclosed above; inserting the distal end of the introducer sheath into the insertion site; enclosing the insertion device within the channel defined between the first elongate protective cover and the second elongate protective cover of the insertion device protection apparatus; positioning the insertion device protection apparatus with the enclosed insertion device proximal to the hub of the introducer sheath; advancing the insertion device enclosed in the insertion device protection apparatus towards the hub and inserting the insertion device enclosed in the insertion device protection apparatus via the hub into the insertion site through the distal end of the introducer sheath, while retaining a proximal end of the first elongate protective cover and a proximal end of the second elongate protective cover of the insertion device protection apparatus external to the hub; and progressively disengaging the first elongate protective cover from the second elongate protective cover of the insertion device protection apparatus enclosing the inserted insertion device by pulling the proximal end of the first elongate protective cover and/or the proximal end of the second elongate protective cover external to the hub to partially or completely expose the inserted insertion device distal to the insertion site.

In an embodiment, the method disclosed herein can additionally comprise one or more of the following structures. In an embodiment, removing the insertion device protection apparatus comprises allowing the two or more interacting parts of the insertion device protection apparatus to partially or completely disengage and fall away from each other. In another embodiment, removing the insertion device protection apparatus comprises partially or completely removing the two or more interacting parts of the insertion device protection apparatus from the insertion device or the insertion assembly comprising the hub and the insertion device.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates a perspective view of an insertion device protection apparatus.

FIG. 2A exemplarily illustrates a perspective view of an insertion system showing an embodiment of the insertion device protection apparatus enclosing an insertion assembly comprising a hub and an insertion device.

FIGS. 2B-2C exemplarily illustrate perspective views of the insertion system, showing two types of insertion devices.

FIG. 3 exemplarily illustrates a perspective view of the insertion system, showing a distal end of the insertion device protection apparatus pressing against a surface of an insertion site.

FIG. 4 illustrates a method for securely inserting an insertion device into an insertion site, while protecting the insertion device from infection and/or damage before and during the insertion of the insertion device into the insertion site.

FIGS. 5A-5C exemplarily illustrate elevation views of the insertion system, showing progressive disengagement of two elongate protective covers of the insertion device protection apparatus from a proximal end of the insertion device protection apparatus for allowing an insertion device to advance towards and be securely inserted into an insertion site.

FIG. 6 illustrates an embodiment of the method for securely inserting an insertion device into an insertion site, while protecting the insertion device from infection and/or damage before and during the insertion of the insertion device into the insertion site.

FIGS. 7A-7D exemplarily illustrate elevation views of the insertion system, showing progressive disengagement and removal of two elongate protective covers of the insertion device protection apparatus from a distal end of the insertion device protection apparatus for allowing an insertion device to advance towards and be securely inserted into an insertion site.

FIGS. 8A-8D exemplarily illustrate elevation views of the insertion system, showing progressive disengagement and removal of two elongate protective covers of the insertion device protection apparatus from a proximal end and a distal end of the insertion device protection apparatus for allowing an insertion device to advance towards an insertion site.

FIG. 9 illustrates an embodiment of the method for securely inserting an insertion device into an insertion site, while protecting the insertion device from infection and/or damage before, during, and after the insertion of the insertion device into the insertion site.

FIGS. 10A-10D exemplarily illustrate elevation views of the insertion system, showing progressive disengagement and removal of two elongate protective covers of the insertion device protection apparatus to expose an insertion device distal to an insertion site, after insertion of the insertion device enclosed in the insertion device protection apparatus into the insertion site.

### DETAILED DESCRIPTION OF THE INVENTION

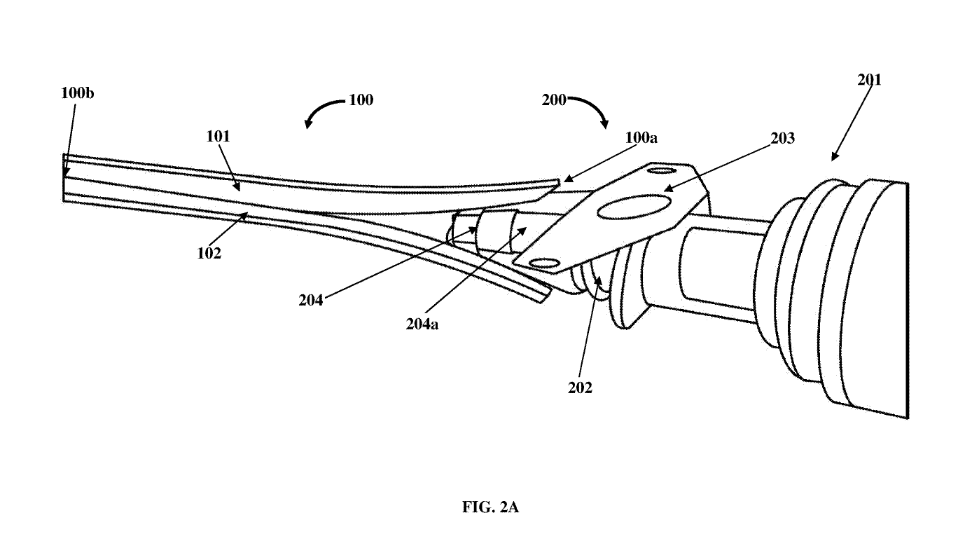

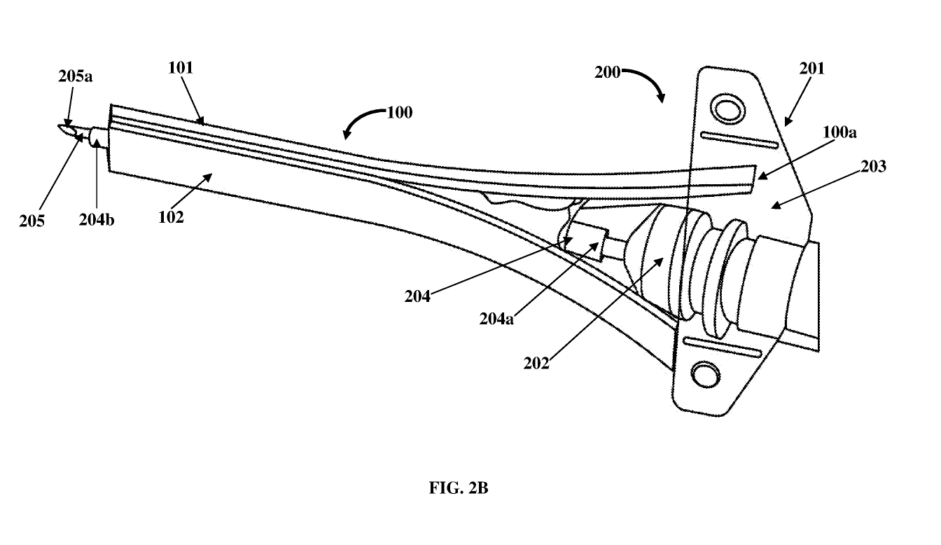

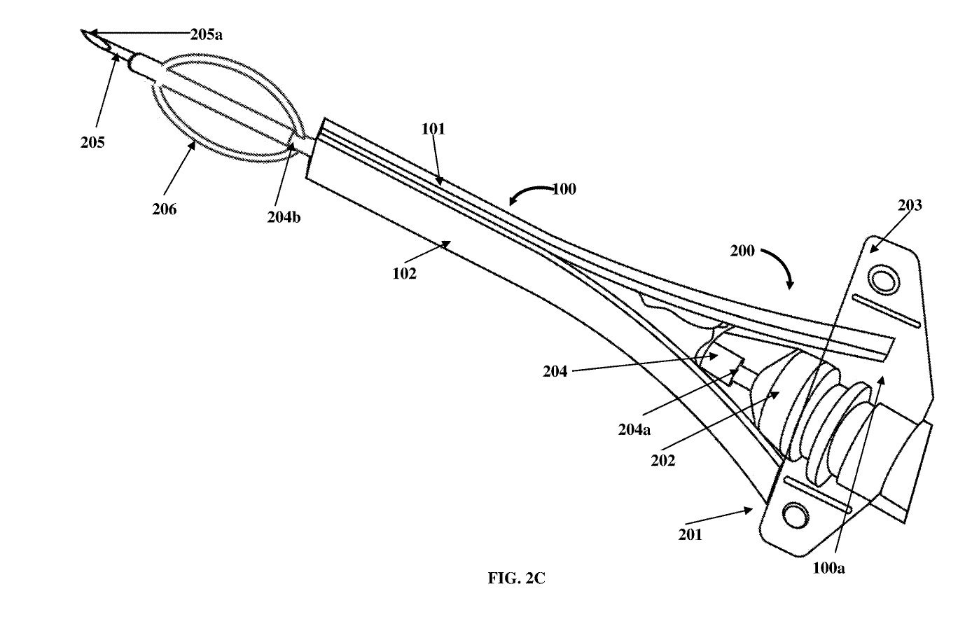

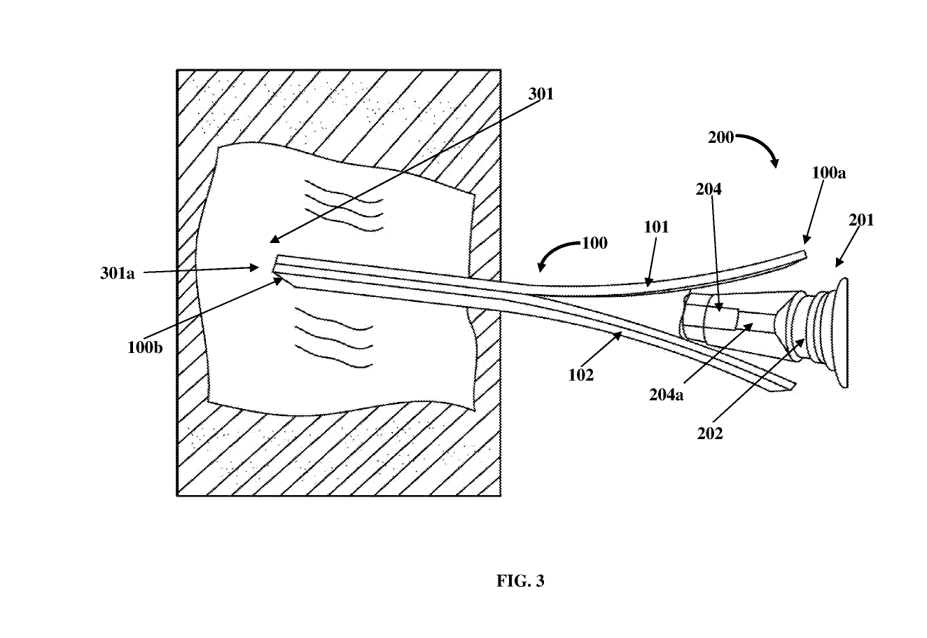

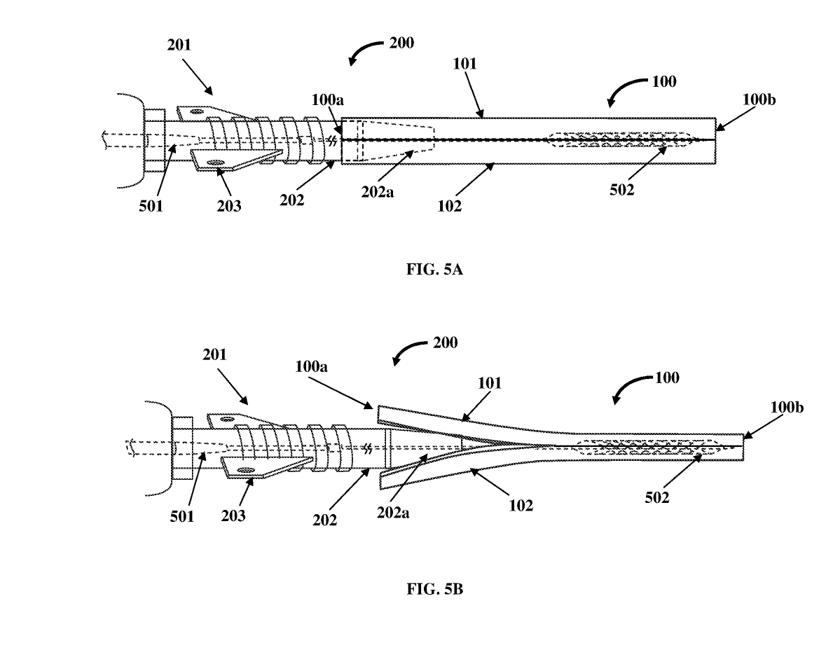

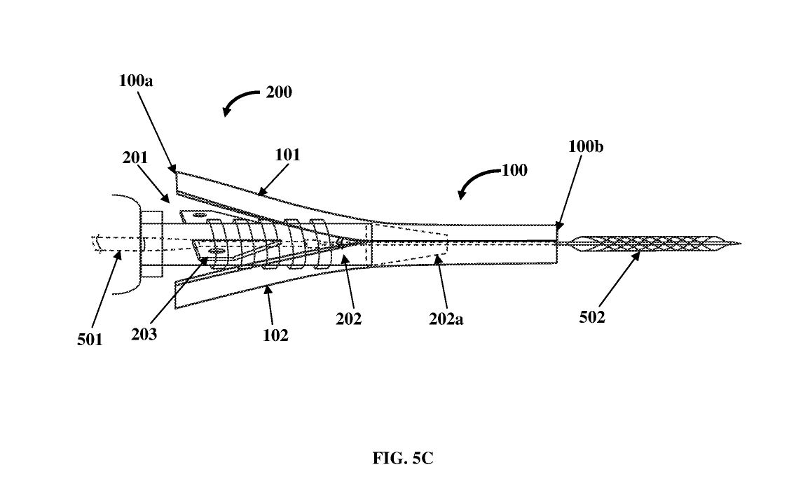

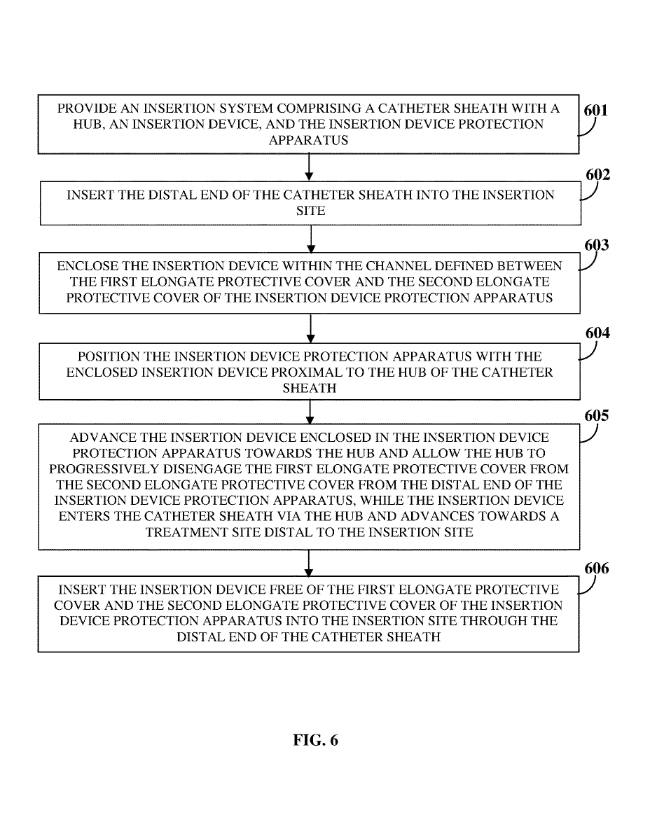

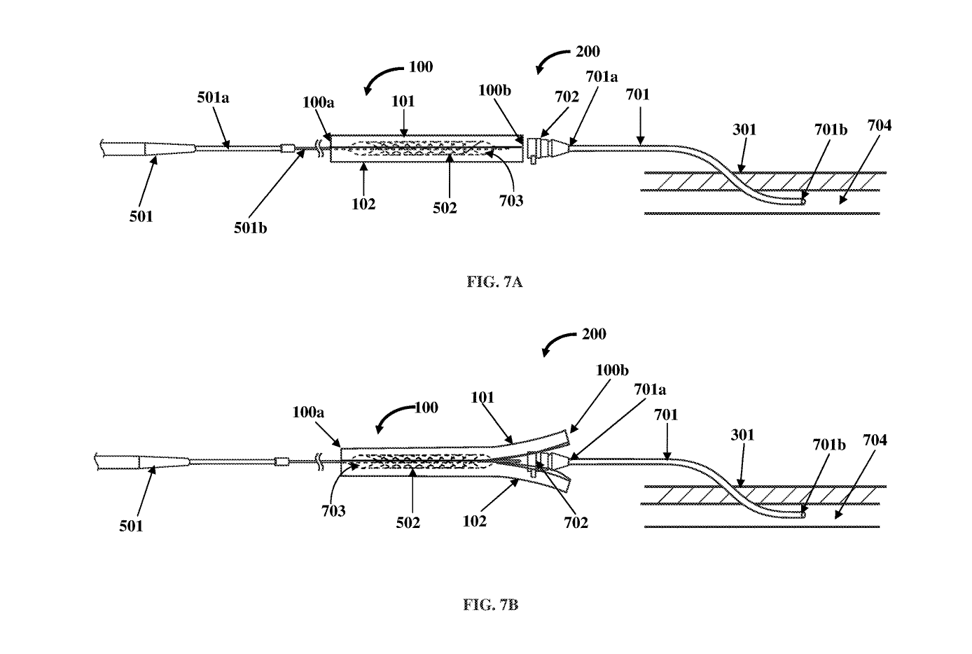

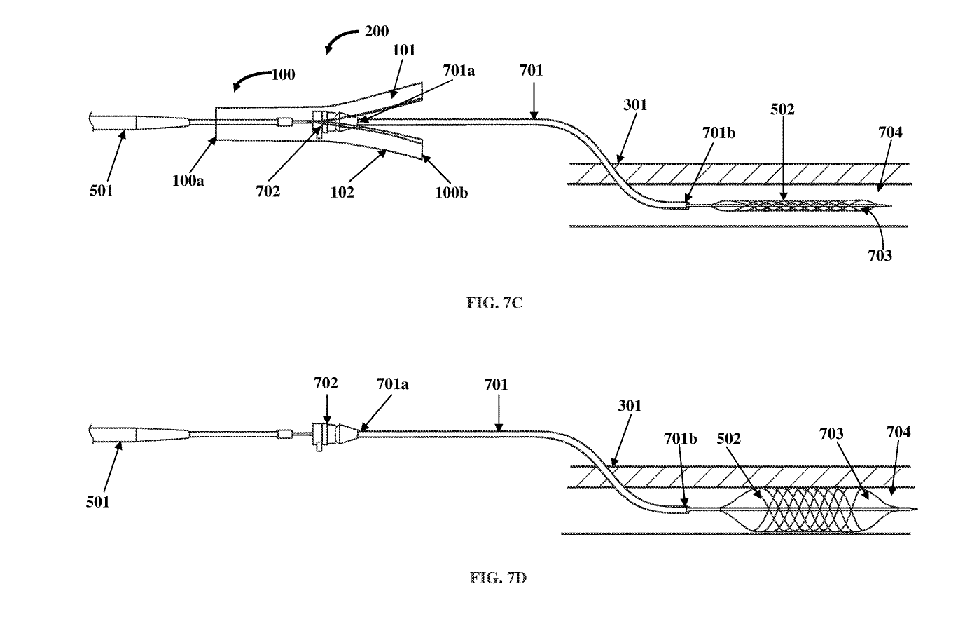

Disclosed herein are insertion devices, apparatuses, systems, and methods for preventing and/or substantially reducing an occurrence of an infection and/or damage and/or contamination to an insertion device before, and/or during, and/or after an insertion of the insertion device into an insertion site 301 exemplarily illustrated in FIG. 3, FIGS. 7A-7D, FIGS. 8A-8D, and FIGS. 10A-10D. As used herein, “insertion device”, in an embodiment, refers to a device that is inserted into an insertion site 301 and used for injecting or infusing fluids or solutions into a treatment site 704 distal to the insertion site 301 exemplarily illustrated in FIGS. 7A-7D, FIGS. 8A-8D, and FIGS. 10A-10D, or for withdrawing fluids out from the treatment site 704. The insertion device is an injection device or an infusion device, for example, a needle, a catheter, a medical balloon, a stent, a balloon catheter, a drug delivery device, a fluid delivery line, etc., or any combination thereof. The insertion device is, for example, a catheter tube 204 with a needle 205 exemplarily illustrated in FIGS. 2A-2B, or a catheter tube 204 with a medical balloon 206 and a needle 205 exemplarily illustrated in FIG. 2C, or a balloon dilation catheter 501 with a stent 502 exemplarily illustrated in FIGS. 5A-5C, or a balloon dilation catheter 501 with a balloon 703 positioned within a stent 502 exemplarily illustrated in FIGS. 7A-7D, FIGS. 8A-8D, and FIGS. 10A-10D.

Also, as used herein, “insertion site” refers to a location, for example, an injection site or an infusion site such as a patient's skin above a treatment site 704 such as a blood vessel on the patient's leg or arm or other body part of the patient needing dilation as exemplarily illustrated in FIGS. 7A-7D, FIGS. 8A-8D, and FIGS. 10A-10D, for inserting an insertion device. Also, as used herein, “treatment site” refers to a location for treatment, for example, dilation of a blood vessel with a balloon catheter, or for placement of a stent 502, or for administration of a medication, etc. The devices, apparatuses, systems, and methods disclosed herein allow for insertion of an insertion device for injections or infusions, or for treatment such as dilation of a blood vessel with a balloon catheter, or for placement of a stent 502, or for administration of a medication in a desired location in a manner that reduces the likelihood of an infection and/or damage and/or contamination to the insertion device and its tip, while reducing the additional force required when using an injection or infusion system with a protective covering. The needles used in the devices, systems, and methods disclosed herein are generally made of metals, for example, stainless steel of the 303, 304, 316, or 400 series, titanium, or other such metals or alloys, and can also be made out of plastic, glass, or ceramic materials depending on the application. The catheters used in the devices, systems, and methods disclosed herein generally include a metal or hard polymer insertion needle or wire and a polymer catheter tube.

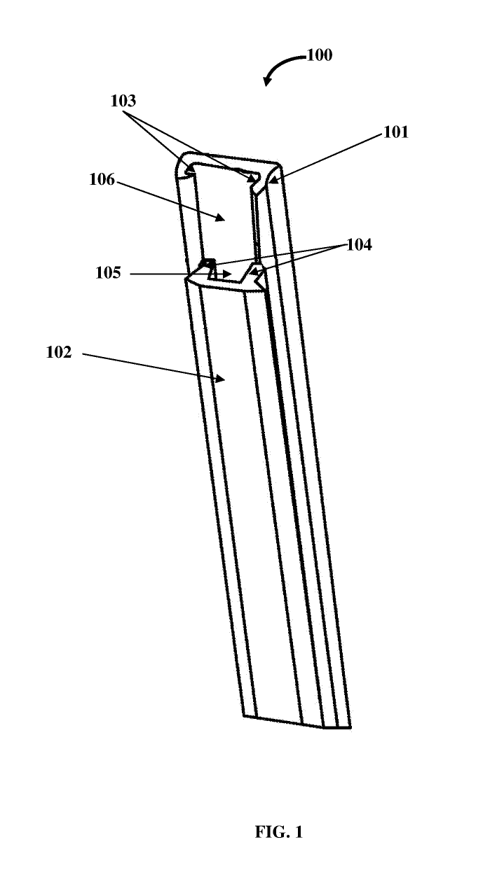

FIG. 1 exemplarily illustrates a perspective view of an insertion device protection apparatus 100 that protects an insertion device, for example, a needle, a catheter, a medical balloon, a stent, a balloon catheter, a drug delivery device, a fluid delivery line, etc., and/or a combination thereof, from infection and/or damage and/or contamination before, and/or during, and/or after insertion of the insertion device into an insertion site 301 exemplarily illustrated in FIG. 3, FIGS. 7A-7D, FIGS. 8A-8D, and FIGS. 10A-10D. In an embodiment, the insertion device protection apparatus 100 is used for covering a metal needle or a plastic needle that is used for injecting fluids, medications, or other materials into intravenous (IV) tubing, butterfly needles, parenteral nutrition tubing, respiratory tubing, urinary tubing, cerebral spinal fluid tubing, or any other medical tubing that is used to deliver fluids or materials to humans or animals. The insertion device protection apparatus 100 can also be used to cover catheters that are used for intravenous, intra-arterial, abdominal, renal, central nervous system or any other access.

The insertion device protection apparatus 100 disclosed herein is a protective covering comprising two interacting parts, namely, a first elongate protective cover 101 and a second elongate protective cover 102. The second elongate protective cover 102 is detachably engaged to the first elongate protective cover 101. The first elongate protective cover 101 and the second elongate protective cover 102 define a channel 105 extending through a length of the insertion device protection apparatus 100 for enclosing an insertion device, for example, a catheter tube 204 with a needle 205 exemplarily illustrated in FIGS. 2A-2B, or a catheter tube 204 with a medical balloon 206 and a needle 205 exemplarily illustrated in FIG. 2C, therewithin before, and/or during, and/or after the insertion of the insertion device into the insertion site 301. The effective diameter of the channel 105 defined between the first elongate protective cover 101 and the second elongate protective cover 102 of the insertion device protection apparatus 100 depends on molding requirements, technical capabilities of a manufacturer of the insertion assembly 201, application, and areas of the insertion device requiring protection. The insertion device is inserted by a user, for example, a medical practitioner into the insertion site 301 of a patient. The first elongate protective cover 101 is progressively disengaged from the second elongate protective cover 102 from a proximal end 100a and/or a distal end 100b of the insertion device protection apparatus 100 before, and/or during, and/or after the insertion of the insertion device into the insertion site 301 as exemplarily illustrated in FIGS. 2A-2C, FIG. 3, FIGS. 5A-5C, FIGS. 7A-7D, FIGS. 8A-8D, and FIGS. 10A-10D.

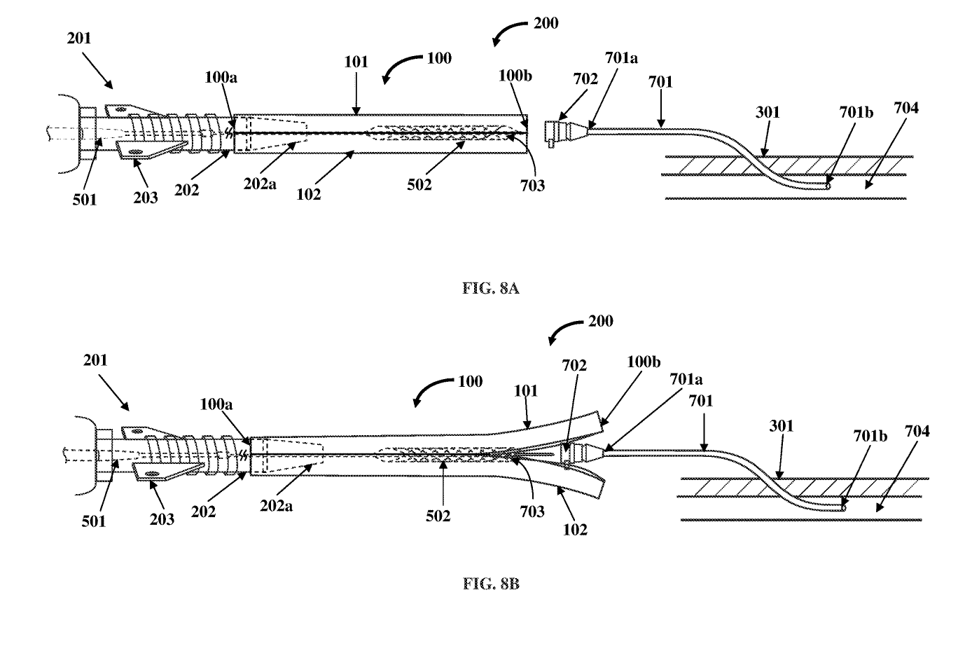

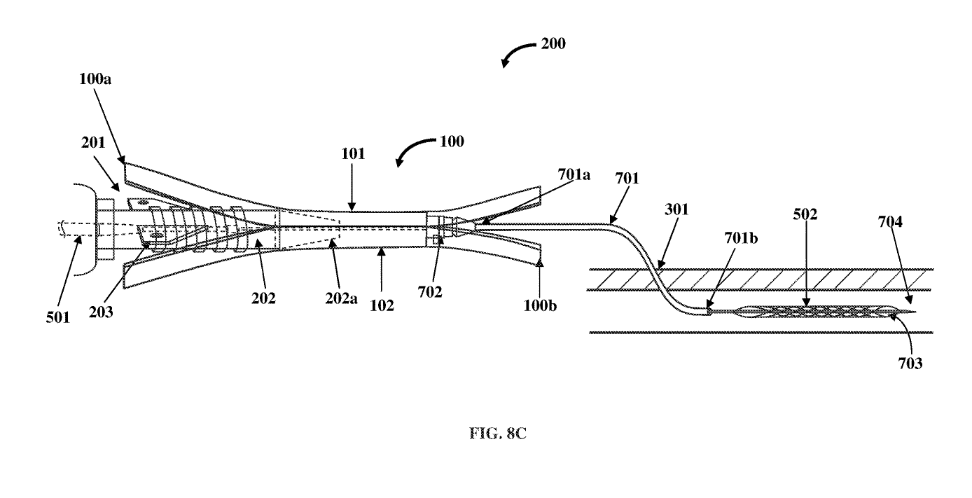

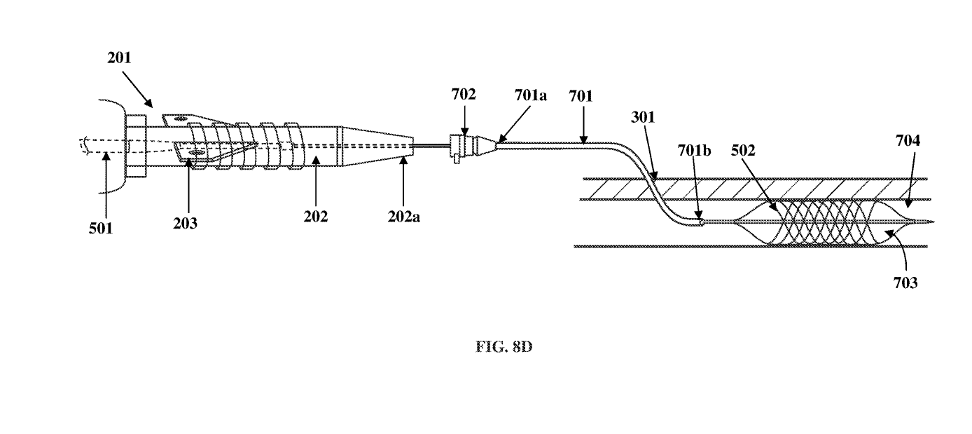

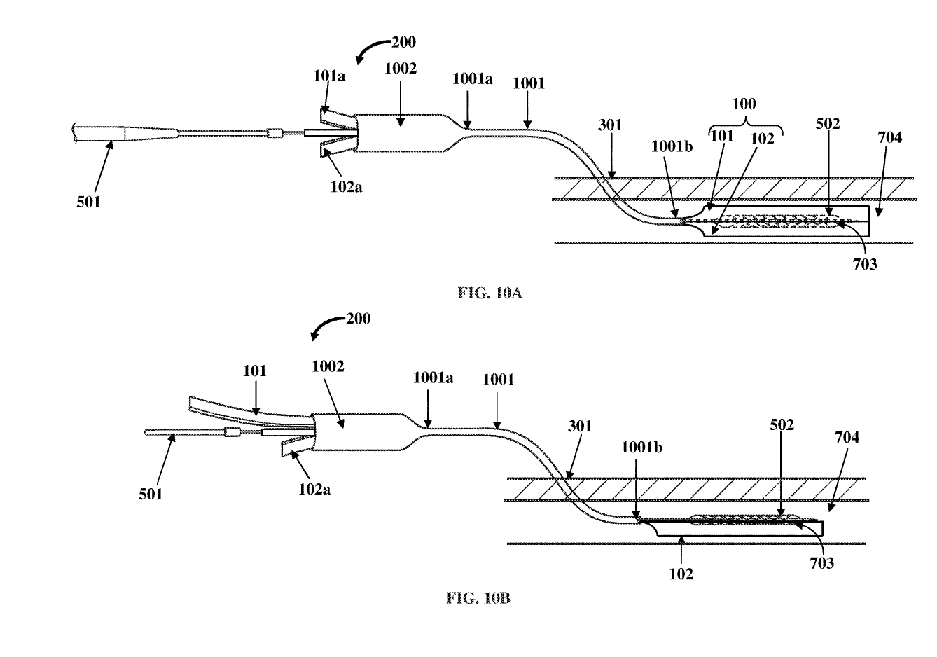

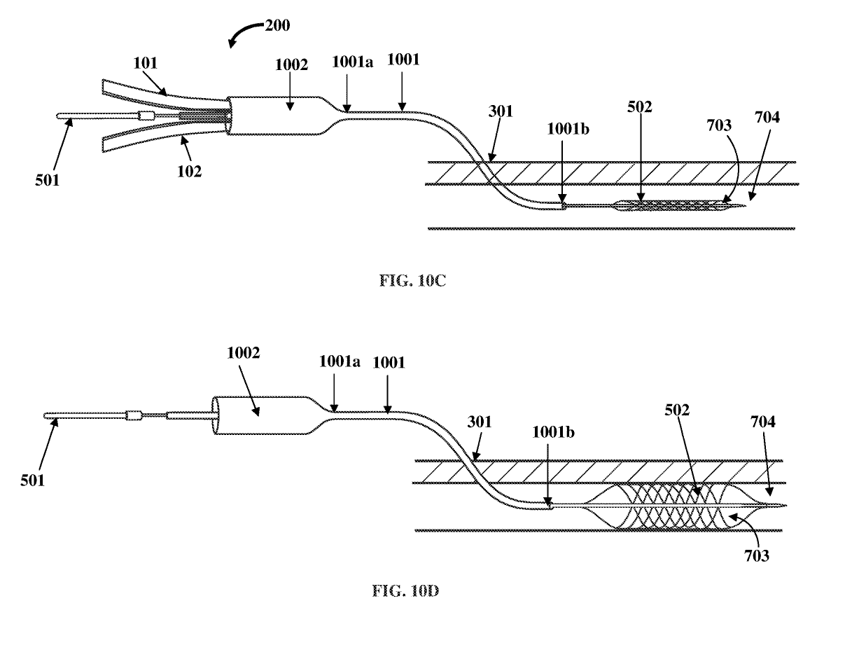

In an embodiment, the first elongate protective cover 101 is progressively disengaged from the second elongate protective cover 102 from the proximal end 100a of the insertion device protection apparatus 100 as exemplarily illustrated in FIGS. 2A-2C, FIG. 3, and FIGS. 5A-5C, for allowing the insertion device to advance towards and be securely inserted into the insertion site 301. In another embodiment, the first elongate protective cover 101 is progressively disengaged from the second elongate protective cover 102 from the distal end 100b of the insertion device protection apparatus 100 as exemplarily illustrated in FIGS. 7A-7D, for allowing the insertion device to advance towards and be securely inserted into the insertion site 301. In another embodiment, the first elongate protective cover 101 is progressively disengaged from the second elongate protective cover 102 from the proximal end 100a and the distal end 100b of the insertion device protection apparatus 100 as exemplarily illustrated in FIGS. 8A-8D, for allowing the insertion device to advance towards and be securely inserted into the insertion site 301. In another embodiment, the first elongate protective cover 101 is progressively disengaged from the second elongate protective cover 102 to expose the insertion device distal to the insertion site 301, after the insertion device enclosed in the insertion device protection apparatus 100 is inserted into the insertion site 301 as exemplarily illustrated in FIGS. 10A-10D.

In an embodiment, the first elongate protective cover 101 is progressively disengaged from the second elongate protective cover 102 external to the insertion site 301, for example, a patient's skin, while the insertion device is advanced towards the insertion site 301 as exemplarily illustrated in FIGS. 7A-7D and FIGS. 8A-8D. In this embodiment, the first elongate protective cover 101 and the second elongate protective cover 102 of the insertion device protection apparatus 100 are progressively disengaged from each other and removed outside the insertion site 301, while the insertion device is advanced towards and securely inserted into the insertion site 301. In another embodiment, the insertion device protection apparatus 100 with the enclosed insertion device is configured to be inserted into the insertion site 301 prior to the progressive disengagement and removal of the first elongate protective cover 101 and the second elongate protective cover 102 to expose the insertion device for use distal to the insertion site 301, for example, at a treatment site 704 such as a blood vessel as exemplarily illustrated in FIGS. 10A-10D. In this embodiment, the insertion device protection apparatus 100 enclosing the insertion device is inserted into the insertion site 301 and advanced towards the treatment site 704, after which the first elongate protective cover 101 and the second elongate protective cover 102 of the insertion device protection apparatus 100 are progressively disengaged from each other at the treatment site 704 and removed from the treatment site 704 to the outside of the patient's body. In this embodiment, the insertion device protection apparatus 100 protects the insertion device from infection and/or damage before, during, and after the insertion of the insertion device into the insertion site 301.

In an embodiment as exemplarily illustrated in FIG. 1, the first elongate protective cover 101 is configured, for example, as a groove portion of the insertion device protection apparatus 100, and the second elongate protective cover 102 is configured, for example, as a protrusion portion of the insertion device protection apparatus 100. In this embodiment, the first elongate protective cover 101 comprises at least two grooves 103, and the second elongate protective cover 102 comprises at least two protrusions 104. The protrusions 104 of the second elongate protective cover 102 slidably engage, and in an embodiment, interlock with the grooves 103 of the first elongate protective cover 101 when the second elongate protective cover 102 is removably inserted into the first elongate protective cover 101. The first elongate protective cover 101 and the second elongate protective cover 102 of the insertion device protection apparatus 100 engage with each other such that the grooves 103 in the first elongate protective cover 101 receive the protrusions 104 of the second elongate protective cover 102.

The materials and design of the grooves 103 and the protrusions 104 of the insertion device protection apparatus 100 allow the grooves 103 and the protrusions 104 to deform and allow the insertion device protection apparatus 100 to form a channel 105 through the length of the insertion device protection apparatus 100. The channel 105 can receive an insertion device, for example, a needle 205 and/or a catheter tube 204 exemplarily illustrated in FIG. 2B, from the proximal end 100a of the insertion device protection apparatus 100. The grooves 103 of the first elongate protective cover 101 progressively disengage from the protrusions 104 of the second elongate protective cover 102 for allowing the insertion device to advance down the insertion device protection apparatus 100 during the insertion of the insertion device into the insertion site 301. In an embodiment, the first elongate protective cover 101 and the second elongate protective cover 102 of the insertion device protection apparatus 100 split and disengage from each other for allowing the insertion device, for example, the needle 205 and/or the catheter tube 204 to progress further down the length of the insertion device protection apparatus 100 during an injection.

The insertion device protection apparatus 100 can be made, for example, of a plastic material. The second elongate protective cover 102 can be made of a material different from the material of the first elongate protective cover 101. Examples of materials used for manufacturing the elongate protective covers 101 and 102 of the insertion device protection apparatus 100 are extra flexible convoluted fluorinated ethylene propylene (FEP), perfluoroalkoxy alkane (PFA), ethylene tetrafluoroethylene (ETFE), polyether ether ketone (PEEK), polyimide, Kynar® fluoropolymers of Arkema, Inc., such as polyvinylidene fluoride, etc. The materials used for manufacturing the elongate protective covers 101 and 102 are compatible with a sterilization process, for example, an ethylene oxide (ETO) sterilization, an autoclave sterilization, a gamma sterilization, etc., and are selected based on nature of a surgical environment. The protrusions 104 of the second elongate protective cover 102 can be made of a material different from portions of the second elongate protective cover 102 that do not interlock with the grooves 103 of the first elongate protective cover 101. The protrusions 104 of the second elongate protective cover 102 can be made of an elastomeric material, for example, silicone. The insertion device protection apparatus 100 can be formed, for example, through an extrusion process, an injection molding process, or any other suitable polymer formation process.

The channel 105 defined between the first elongate protective cover 101 and the second elongate protective cover 102 can be uniform in dimension across the length of the insertion device protection apparatus 100. In an embodiment, the channel 105 tapers in depth, width, or both depth and width across the length of the insertion device protection apparatus 100. The first elongate protective cover 101 and the second elongate protective cover 102 of the insertion device protection apparatus 100 can be of the same length or of different lengths. As exemplarily illustrated in FIG. 1, the length of one of the elongate protective covers 101 and 102, for example, the second elongate protective cover 102 of the insertion device protection apparatus 100 is substantially shorter than the length of the first elongate protective cover 101. The difference in lengths creates a cavity 106 proximal to the channel 105. The cavity 106 enables a user, for example, a medical practitioner to position an insertion device easily, swiftly, and securely into the channel 105 of the insertion device protection apparatus 100. In an embodiment, the length of one of the elongate protective covers 101 and 102, for example, the second elongate protective cover 102 of the insertion device protection apparatus 100 is about 10% less than the length of the first elongate protective cover 101.

FIG. 2A exemplarily illustrates a perspective view of an insertion system 200 showing an embodiment of the insertion device protection apparatus 100 enclosing an insertion assembly 201 comprising a hub 202 and an insertion device, for example, a catheter tube 204. The insertion assembly 201 of the insertion system 200 is inserted into the insertion device protection apparatus 100 comprising at least two elongate protective covers 101 and 102. The catheter tube 204 extends from the hub 202 of the insertion assembly 201 for insertion into an insertion site 301 exemplarily illustrated in FIG. 3. The catheter tube 204 is made of any suitable material that is biocompatible, for example, a material that does not cause thrombosis or irritation at the insertion site 301. The two elongate protective covers 101 and 102 of the insertion device protection apparatus 100 are in engageable communication with each other and enclose the hub 202 and the catheter tube 204 of the insertion assembly 201.

In an embodiment, the insertion assembly 201 disclosed herein further comprises a winged element 203 fixedly attached to the hub 202 and positioned proximal to the catheter tube 204 for allowing a user to position and affix the catheter tube 204 into the insertion site 301. The catheter tube 204 of the insertion assembly 201 at its proximal end 204a is connected to the hub 202 and the winged element 203. In an embodiment, an effective diameter of the channel 105 defined between the first elongate protective cover 101 and the second elongate protective cover 102 of the insertion device protection apparatus 100 exemplarily illustrated in FIG. 1, is less than a diameter of the hub 202 of the insertion assembly 201. For example, the effective diameter of the channel 105 defined between the first elongate protective cover 101 and the second elongate protective cover 102 of the insertion device protection apparatus 100 is about one third the diameter of the hub 202 of the insertion assembly 201. The catheter tube 204 fits into the channel 105. As exemplarily illustrated in FIG. 2A, the insertion assembly 201 is shown in a pre-insertion configuration with the insertion assembly 201 partially inserted into the insertion device protection apparatus 100. The pre-insertion configuration refers to the configuration of the insertion system 200 comprising the insertion device protection apparatus 100 enclosing the insertion assembly 201 prior to insertion of the insertion device into the insertion site 301. The proximal end 204a of the catheter tube 204 is located at the proximal end 100a of the insertion device protection apparatus 100. The winged element 203 of the insertion assembly 201 facilitates positioning of the catheter tube 204 into the insertion site 301 for affixing a catheter assembly into a patient as practiced in the medical arts. In use, the winged element 203 is positioned near a patient's skin when the catheter tube 204 is inserted into the insertion site 301.

FIGS. 2B-2C exemplarily illustrate perspective views of the insertion system 200, showing two types of insertion devices, for example, a catheter tube 204 with a needle 205, and a catheter tube 204 with a combination of a medical balloon 206 and a needle 205. FIG. 2B shows the distal end 204b of the catheter tube 204 with a needle 205 protruding from the distal end 204b. FIG. 2C shows a medical balloon 206 and a needle 205 protruding from the distal end 204b of the catheter tube 204. The needle 205 is, for example, made of 420 series stainless steel. The needle 205 or the combination of the medical balloon 206 and the needle 205 protruding from the distal end 204b of the catheter tube 204 and a majority of the catheter tube 204 are enclosed and protected by the insertion device protection apparatus 100 prior to advancement of the catheter tube 204 towards the insertion site (not shown in FIGS. 2B-2C) as exemplarily illustrated in FIG. 2A, and is inserted into the insertion site 301 exemplarily illustrated in FIG. 3, on reaching the insertion site 301.

The proximal end 204a of the catheter tube 204 is in communication with and adjacent to the hub 202 of the insertion assembly 201. The hub 202 is in communication with and adjacent to the winged element 203 which is located proximal to a section of the insertion device protection apparatus 100 where the two elongate protective covers 101 and 102 of the insertion device protection apparatus 100 separate. The insertion device protection apparatus 100 splits to allow the insertion assembly 201 to progress further down the channel 105 defined between the elongate protective covers 101 and 102 of the insertion device protection apparatus 100 exemplarily illustrated in FIG. 1. In an embodiment, only a tip 205a of the insertion device, for example, the needle 205 exemplarily illustrated in FIG. 2B, and not the entire insertion device, is enclosed within the channel 105 defined between the elongate protective covers 101 and 102 of the insertion device protection apparatus 100. In an embodiment, only the medical balloon 206 and the tip 205a of the needle 205 exemplarily illustrated in FIG. 2C, and not the entire insertion device are enclosed within the channel 105 defined between the elongate protective covers 101 and 102 of the insertion device protection apparatus 100.

FIG. 3 exemplarily illustrates a perspective view of the insertion system 200, showing a distal end 100b of the insertion device protection apparatus 100 pressing against a surface 301a of an insertion site 301. The distal end 204b of the catheter tube 204 exemplarily illustrated in FIGS. 2B-2C, is beneath the surface 301a of the insertion site 301 and therefore not shown in FIG. 3. The winged element 203 of the insertion assembly 201 exemplarily illustrated in FIGS. 2A-2C, is also not shown in FIG. 3, because of the partial view of the insertion assembly 201 shown in FIG. 3. In FIG. 3, the proximal end 204a of the catheter tube 204 is more easily seen as the proximal end 204a approaches the middle of the insertion device protection apparatus 100. As the insertion assembly 201 is inserted into the insertion site 301, the insertion device protection apparatus 100 splits further. Once the catheter tube 204 is fully inserted, the insertion device protection apparatus 100 falls fully apart and separates from the insertion assembly 201 and a user, for example, a medical practitioner can remove the insertion device protection apparatus 100.

The first elongate protective cover 101 of the insertion device protection apparatus 100 can slide relative to the second elongate protective cover 102. For example, if the second elongate protective cover 102 is held in place and a force is exerted on the first elongate protective cover 101 along the longitudinal axis of the first elongate protective cover 101, the first elongate protective cover 101 slides. Conversely, the second elongate protective cover 102 and the first elongate protective cover 101 of the insertion device protection apparatus 100 do not easily separate in any direction off the longitudinal axis. The channel 105 defined between the first elongate protective cover 101 and the second elongate protective cover 102 is smaller in effective cross-section than the cross-section of the hub 202 of the insertion assembly 201, such that when the hub 202 travels along the length of the insertion device protection apparatus 100, the second elongate protective cover 102 and the first elongate protective cover 101 split apart and separate from each other. The size of the channel 105 relative to the size of the hub 202 can be selected so that as the hub 202 approaches the surface 301a of the insertion site 301, the first elongate protective cover 101 and the second elongate protective cover 102 of the insertion device protection apparatus 100 split apart from each other.

The first elongate protective cover 101 and the second elongate protective cover 102 of the insertion device protection apparatus 100 that separate after insertion of the needle 205 or the catheter tube 204 into the insertion site 301 avoid contamination of the insertion site 301 by the insertion device protection apparatus 100. The insertion device protection apparatus 100 disclosed herein can also be adjusted for the amount of force needed to separate the first elongate protective cover 101 and the second elongate protective cover 102 of the insertion device protection apparatus 100 in view of the size of the needle 205 or the catheter tube 204, or the hub 202 of the insertion assembly 201. In this way, an injection or infusion insertion assembly 201 can be used with the insertion device protection apparatus 100 in a delicate area of a patient's body or with a sensitive patient without application of a large amount of force at the insertion site 301.

For purposes of illustration, the detailed description refers to the insertion device being the needle 205 or the catheter tube 204 as exemplarily illustrated in FIG. 2B; however the scope of the method and the insertion system 200 disclosed herein is not limited to the insertion device being the needle 205 or the catheter tube 204, but may be extended to include a medical balloon 206 and a needle 205 exemplarily illustrated in FIG. 2C, a stent 502 exemplarily illustrated in FIGS. 5A-5C, FIGS. 7A-7D, FIGS. 8A-8D, and FIGS. 10A-10D, a drug delivery device, a fluid delivery line, and other functionally equivalent structures. The two elongate protective covers 101 and 102 of the insertion device protection apparatus 100 are positioned around the insertion device, for example, the needle 205, the stent 502, the medical balloon 206 with the needle 205, or another insertion device that is to be inserted into the insertion site 301, to prevent infection and/or damage and/or contamination to the insertion device before, and/or during, and/or after the insertion of the insertion device into the insertion site 301. The two elongate protective covers 101 and 102 can also be used to protect delicate catheters, for example, dye catheters, balloon catheters, etc., from infection and/or damage and/or contamination before, and/or during, and/or after the insertion of the catheter into the insertion site 301.

The insertion device protection apparatus 100 protects the insertion device both before and during insertion, that is, typically before entering the insertion site 301, for example, a patient's skin, and in an embodiment, also protects the insertion device distal to the insertion site 301, for example, at a treatment site 704 inside the patient's body, such as, in a blood vessel as exemplarily illustrated in FIGS. 10A-10D, from infection and/or damage and/or contamination until the insertion device reaches its desired location, after which the insertion device protection apparatus 100 is retracted. In an embodiment, the two elongate protective covers 101 and 102 of the insertion device protection apparatus 100 are retracted after positioning the insertion device, for example, the stent 502, the medical balloon 206 with the needle 205, or other insertion device at the treatment site 704 distal to the insertion site 301. For example, a stent 502 attached to a balloon dilation catheter 501 exemplarily illustrated in FIGS. 10A-10D, is positioned inside the insertion device protection apparatus 100, that is, in the channel 105 between the two elongate protective covers 101 and 102 of the insertion device protection apparatus 100 exemplarily illustrated in FIG. 1. The insertion device protection apparatus 100 enclosing the stent 502 and a portion of the balloon dilation catheter 501 is inserted into an insertion site 301, for example, a patient's skin, and after reaching a desired location, for example, a treatment site 704 such as a blood vessel or other structure of a patient needing dilation, the elongate protective covers 101 and 102 are pulled back or slidably disengaged from each other and retracted from the blood vessel, exposing the stent 502 and the portion of the balloon dilation catheter 501 inside the blood vessel as disclosed in the detailed description of FIGS. 10A-10D.

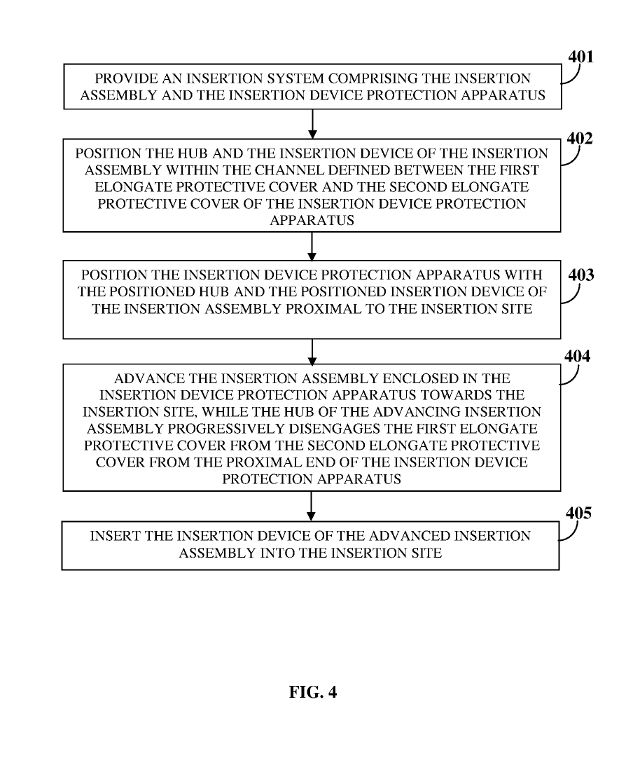

FIG. 4 illustrates a method for securely inserting an insertion device, for example, a balloon dilation catheter 501 with a stent 502 exemplarily illustrated in FIGS. 5A-5C, into an insertion site 301 exemplarily illustrated in FIG. 3, while protecting the insertion device from infection and/or damage and/or contamination before and during the insertion of the insertion device into the insertion site 301. An insertion system 200 comprising the insertion assembly 201 and the insertion device protection apparatus 100 as disclosed in the detailed description of FIG. 1 and FIGS. 2A-2C, is provided 401. The method disclosed herein allows the insertion assembly 201 to advance towards an insertion site 301 with the insertion device enclosed within the insertion device protection apparatus 100 before and during an injection. A user, for example, a medical practitioner, positions 402 the hub 202 and the insertion device of the insertion assembly 201 within the channel 105 defined between the first elongate protective cover 101 and the second elongate protective cover 102 of the insertion device protection apparatus 100 exemplarily illustrated in FIG. 1. The tip of the insertion device is enclosed within the defined channel 105. The user inserts the insertion device and the hub 202 of the insertion assembly 201 into the insertion device protection apparatus 100 from the proximal end 100a of the insertion device protection apparatus 100 exemplarily illustrated in FIG. 2A. The channel 105 defined between the first elongate protective cover 101 and the second elongate protective cover 102 of the insertion device protection apparatus 100 receives the insertion device from the proximal end 100a of the insertion device protection apparatus 100. The user positions 403 the insertion device protection apparatus 100 with the positioned hub 202 and the positioned insertion device of the insertion assembly 201 proximal to the insertion site 301.

The user advances 404 the insertion assembly 201 enclosed in the insertion device protection apparatus 100 towards the insertion site 301, while the hub 202 of the advancing insertion assembly 201 progressively disengages the first elongate protective cover 101 from the second elongate protective cover 102 from the proximal end 100a of the insertion device protection apparatus 100. That is, the hub 202 travels along the length of the insertion device protection apparatus 100 and splits or separates the first elongate protective cover 101 from the second elongate protective cover 102 starting from the proximal end 100a of the insertion device protection apparatus 100. The user inserts 405 the insertion device of the advanced insertion assembly 201 into the insertion site 301. In an embodiment, the user then completely disengages and removes the first elongate protective cover 101 from the second elongate protective cover 102 of the insertion device protection apparatus 100 that encloses the advanced insertion assembly 201, after the insertion device of the advanced insertion assembly 201 is completely inserted into the insertion site 301. In an embodiment, the user removes the insertion device protection apparatus 100 by allowing the first elongate protective cover 101 to disengage and fall away from the second elongate protective cover 102.

FIGS. 5A-5C exemplarily illustrate elevation views of the insertion system 200, showing progressive disengagement of two elongate protective covers 101 and 102 of the insertion device protection apparatus 100 from a proximal end 100a of the insertion device protection apparatus 100 for allowing an insertion device, for example, a balloon dilation catheter 501 with a stent 502 to advance towards and be securely inserted into an insertion site 301 exemplarily illustrated in FIG. 3. The insertion system 200 exemplarily illustrated in FIGS. 5A-5C, comprises the insertion assembly 201 and the insertion device protection apparatus 100. The insertion assembly 201 comprises a hub 202 and an insertion device, for example, a balloon dilation catheter 501 with a stent 502. The hub 202 and the stent 502 extending from the hub 202 of the insertion assembly 201 are enclosed and protected within the channel 105 defined between the elongate protective covers 101 and 102 of the insertion device protection apparatus 100 exemplarily illustrated in FIG. 1. The insertion assembly 201 further comprises a winged element 203 fixedly attached to the hub 202. In FIG. 5A, the insertion assembly 201 is shown in a pre-insertion configuration with the insertion assembly 201 partially inserted into the insertion device protection apparatus 100. In the pre-insertion configuration, the distal end 202a of the hub 202 of the insertion assembly 201 is positioned at the proximal end 100a of the insertion device protective apparatus 100, and the stent 502 of the insertion assembly 201 is positioned at the distal end 100b of the insertion device protective apparatus 100 as exemplarily illustrated in FIG. 5A.

A user, for example, a medical practitioner, advances the insertion assembly 201 such that the hub 202 of the insertion assembly 201 applies pressure against the proximal end 100a of the insertion device protection apparatus 100 prior to insertion of the balloon dilation catheter 501 with the stent 502 into the insertion site 301. The pressure applied at the proximal end 100a of the insertion device protection apparatus 100 due to the advancement of the insertion assembly 201 results in progressive disengagement of the first elongate protective cover 101 from the second elongate protective cover 102 of the insertion device protection apparatus 100 as exemplarily illustrated in FIG. 5B. The disengaged elongate protective covers 101 and 102 of the insertion device protection apparatus 100 slide over the insertion assembly 201 to allow the hub 202 and the balloon dilation catheter 501 with the stent 502 of the insertion assembly 201 to progress further down the channel 105 defined between the elongate protective covers 101 and 102, towards the insertion site 301.

FIG. 5C exemplarily illustrates an elevation view of the insertion system 200, showing the stent 502 attached to the balloon dilation catheter 501 of the insertion assembly 201, protruding from the distal end 100b of the insertion device protection apparatus 100. In this view, the majority of the insertion assembly 201 is partially enclosed within the insertion device protection apparatus 100. The winged element 203 in communication with and adjacent to the hub 202 is located proximal to a section of the insertion device protection apparatus 100 where the two elongate protective covers 101 and 102 of the insertion device protection apparatus 100 separate as exemplarily illustrated in FIG. 5C. In an embodiment, the winged element 203 facilitates the disengagement of the first elongate protective cover 101 from the second elongate protective cover 102 of the insertion device protection apparatus 100.

FIG. 6 illustrates an embodiment of the method for securely inserting an insertion device, for example, a balloon dilation catheter 501 with a stent 502 and a balloon 703 into an insertion site 301 exemplarily illustrated in FIG. 7A, while protecting the insertion device from infection and/or damage before and during the insertion of the insertion device into the insertion site 301. In this embodiment, an insertion system 200 comprising an introducer sheath, for example, a catheter sheath 701 with a hub 702 exemplarily illustrated in FIGS. 7A-7D, an insertion device, and the insertion device protection apparatus 100, is provided 601. A user, for example, a medical practitioner, inserts 602 a distal end 701b of the catheter sheath 701 into the insertion site 301. The user encloses 603 the insertion device within the channel 105 defined between the first elongate protective cover 101 and the second elongate protective cover 102 of the insertion device protection apparatus 100 exemplarily illustrated in FIG. 1. The tip of the insertion device is enclosed within the defined channel 105. The first elongate protective cover 101 and the second elongate protective cover 102 of the insertion device protection apparatus 100 are positioned to enclose the insertion device. The user positions 604 the insertion device protection apparatus 100 with the enclosed insertion device proximal to the hub 702 of the catheter sheath 701.

The user advances 605 the insertion device enclosed in the insertion device protection apparatus 100 towards the hub 702 of the catheter sheath 701 and allows the hub 702 to progressively disengage the first elongate protective cover 101 from the second elongate protective cover 102 from the distal end 100b of the insertion device protection apparatus 100, while the insertion device enters the catheter sheath 701 via the hub 702 and advances towards a treatment site 704 distal to the insertion site 301 exemplarily illustrated in FIGS. 7A-7D. A resultant pressure due to the advancement of the insertion device enclosed by the insertion device protection apparatus 100 at the point of contact between the distal end 100b of the insertion device protection apparatus 100 and the hub 702 of the catheter sheath 701 disengages the two elongate protective covers 101 and 102 from each other. The user inserts 606 the insertion device free of the first elongate protective cover 101 and the second elongate protective cover 102 of the insertion device protection apparatus 100 into the insertion site 301 through the distal end 701b of the catheter sheath 701. The user then advances the insertion device towards the treatment site 704. In an embodiment, the user completely disengages and removes the first elongate protective cover 101 from the second elongate protective cover 102 of the insertion device protection apparatus 100, after the advanced insertion device is completely inserted into the insertion site 301.

Consider an example where an angioplasty procedure is performed on a patient to open blocked arteries and to restore normal blood flow to the heart of the patient using an insertion device, for example, a balloon dilation catheter 501 with a balloon 703 and/or a stent 502 exemplarily illustrated in FIGS. 7A-7D. In this example, a physician prepares the patient for the angioplasty procedure and locates an insertion site 301, for example, the patient's skin above a femoral artery. The physician inserts a needle, for example, a Seldinger needle into the femoral artery. The physician inserts a guidewire into the femoral artery through the inserted Seldinger needle and then removes the inserted Seldinger needle from the femoral artery. The physician inserts an introducer sheath, for example, a catheter sheath 701 exemplarily illustrated in FIGS. 7A-7D, along with a dilator into the femoral artery along the guidewire. The physician then removes the dilator and the guidewire, leaving the catheter sheath 701 inside the femoral artery.

The physician encloses the insertion device in the insertion device protection apparatus 100 and positions the insertion device enclosed by the insertion device protection apparatus 100 proximal to the hub 702 of the inserted catheter sheath 701 exemplarily illustrated in FIGS. 7A-7D. The insertion device protection apparatus 100 protects the insertion device from infection and/or damage and/or contamination before and during the insertion of the insertion device into the femoral artery. The physician advances the insertion device enclosed by the insertion device protection apparatus 100 towards the hub 702 of the catheter sheath 701 and allows the hub 702 to progressively disengage the first elongate protective cover 101 from the second elongate protective cover 102 starting from the distal end 100b of the insertion device protection apparatus 100, thereby allowing the insertion device to enter the catheter sheath 701 via the hub 702 and thereafter advance towards and enter the femoral artery via the distal end 701b of the catheter sheath 701, uninfected, and/or undamaged, and/or uncontaminated. The two elongate protective covers 101 and 102 of the insertion device protection apparatus 100 are completely disengaged proximal to the hub 702 and external to the patient's skin, after the advanced insertion device is completely inserted into the femoral artery. The physician uses the insertion device to open blocked arteries to restore normal blood flow to the heart of the patient.

FIGS. 7A-7D exemplarily illustrate elevation views of the insertion system 200, showing progressive disengagement and removal of two elongate protective covers 101 and 102 of the insertion device protection apparatus 100 from a distal end 100b of the insertion device protection apparatus 100 for allowing an insertion device to advance towards and be securely inserted into an insertion site 301. The insertion site 301 is, for example, a patient's skin 301 above a treatment site 704 such as a blood vessel on the patient's leg or arm or other body part of the patient needing dilation. The treatment site 704 is herein referred to as the “blood vessel” and referenced by the numeral 704. The insertion device is, for example, a balloon dilation catheter 501 with a balloon 703 positioned within a stent 502 as exemplarily illustrated in FIGS. 7A-7D. The balloon dilation catheter 501 comprises a proximal shaft 501a having a diameter of, for example, about 2.4 French (Fr), that is, 0.8 millimeters (mm), and a distal shaft 501b having a diameter of, for example, about 3.6 Fr, that is, 1.2 mm as exemplarily illustrated in FIG. 7A.

A user, for example, a physician, encloses the stent 502 with the balloon 703 extending from the balloon dilation catheter 501 within the channel 105 defined between the first elongate protective cover 101 and the second elongate protective cover 102 of the insertion device protection apparatus 100 exemplarily illustrated in FIG. 1. The physician positions the balloon dilation catheter 501 with the balloon 703 and the stent 502 enclosed by the insertion device protection apparatus 100 proximal to a hub 702 of a catheter sheath 701 as exemplarily illustrated in FIG. 7A. The catheter sheath 701 comprises a proximal end 701a connected to the hub 702 and a distal end 701b that is inserted into the blood vessel 704 as exemplarily illustrated in FIGS. 7A-7D. The physician positions the hub 702 of the catheter sheath 701 proximal to the patient's leg or arm or other body part of the patient needing dilation. The physician inserts the distal end 701b of the catheter sheath 701 into the blood vessel 704 of the patient.

The physician thereafter advances the balloon dilation catheter 501 with the stent 502 and the balloon 703 enclosed by the insertion device protection apparatus 100 towards the hub 702 of the catheter sheath 701 and allows the hub 702 to progressively disengage the first elongate protective cover 101 from the second elongate protective cover 102 starting from the distal end 100b of the insertion device protection apparatus 100 as exemplarily illustrated in FIG. 7B, while the balloon dilation catheter 501 with the stent 502 and the balloon 703 enters the catheter sheath 701 via the hub 702 and advances towards the blood vessel 704. When the insertion device protection apparatus 100 enclosing the advancing balloon dilation catheter 501 with the stent 502 and the balloon 703 contacts the hub 702, a resultant pressure on the insertion device protection apparatus 100 at the point of contact between the distal end 100b of the insertion device protection apparatus 100 and the hub 702 of the catheter sheath 701, due to the advancement of the balloon dilation catheter 501 with the stent 502 and the balloon 703, disengages the first elongate protective cover 101 from the second elongate protective cover 102 from the distal end 100b of the insertion device protection apparatus 100, allowing the balloon dilation catheter 501 with the stent 502 and the balloon 703 to enter the catheter sheath 701 uninfected and/or undamaged and/or uncontaminated.

As the two elongate protective covers 101 and 102 of the insertion device protection apparatus 100 are disengaged, the stent 502 with the balloon 703 enclosed within the channel 105 defined between the two elongate protective covers 101 and 102 of the insertion device protection apparatus 100 is inserted into the hub 702 of the catheter sheath 701 due to the advancement of the balloon dilation catheter 501 by the physician, and towards the area of the blood vessel 704 requiring treatment. As exemplarily illustrated in FIG. 7C, the disengaged elongate protective covers 101 and 102 slide over the hub 702 of the catheter sheath 701 as the stent 502 with the balloon 703 progresses through the catheter sheath 701 towards the blood vessel 704 due to the advancement of the balloon dilation catheter 501. The stent 502 with the balloon 703 free of the first elongate protective cover 101 and the second elongate protective cover 102 of the insertion device protection apparatus 100 progresses along the catheter sheath 701 into the blood vessel 704 through the distal end 701b of the catheter sheath 701. The physician removes the first elongate protective cover 101 and the second elongate protective cover 102 of the insertion device protection apparatus 100 after the stent 502 with the balloon 703 is completely inserted into the insertion site 301. The physician further advances the balloon dilation catheter 501 with the stent 502 and the balloon 703 inside the blood vessel 704 until a desired location is reached. The physician positions the stent 502 inside the blood vessel 704 by inflating the balloon 703 using the balloon dilation catheter 501 as exemplarily illustrated in FIG. 7D. The physician then deflates the balloon 703 using the balloon dilation catheter 501 and removes the balloon 703 from the blood vessel 704 leaving the stent 502 inside the blood vessel 704 of the patient.

FIGS. 8A-8D exemplarily illustrate elevation views of the insertion system 200, showing progressive disengagement and removal of two elongate protective covers 101 and 102 of the insertion device protection apparatus 100 from a proximal end 100a and a distal end 100b of the insertion device protection apparatus 100 for allowing an insertion device to advance towards an insertion site 301. The insertion site 301 is, for example, a patient's skin 301 above a treatment site 704 such as a blood vessel on the patient's leg or arm or other body part of the patient needing dilation. The treatment site 704 is herein referred to as the “blood vessel” and referenced by the numeral 704. The insertion device is, for example, a balloon dilation catheter 501 with a balloon 703 positioned within a stent 502 as exemplarily illustrated in FIGS. 8A-8D. The insertion system 200 exemplarily illustrated in FIGS. 8A-8D, comprises the insertion assembly 201 as disclosed in the detailed description of FIGS. 5A-5C, an introducer sheath, for example, a catheter sheath 701 with a hub 702 as disclosed in the detailed description of FIGS. 7A-7D, and the insertion device protection apparatus 100. A user, for example, a physician, encloses the hub 202 and the stent 502 with the balloon 703 extending from the hub 202 of the insertion assembly 201 within the channel 105 defined between the elongate protective covers 101 and 102 of the insertion device protection apparatus 100 exemplarily illustrated in FIG. 1. The distal end 202a of the hub 202 of the insertion assembly 201 is positioned at the proximal end 100a of the insertion device protective apparatus 100, and the stent 502 with the balloon 703 of the insertion assembly 201 is positioned at the distal end 100b of the insertion device protective apparatus 100 as disclosed in the detailed description of FIG. 5A.

The physician positions the hub 202 and the balloon dilation catheter 501 with the stent 502 and the balloon 703 enclosed in the insertion device protection apparatus 100 proximal to the hub 702 of the catheter sheath 701 as exemplarily illustrated in FIG. 8A. The physician positions the hub 702 of the catheter sheath 701 proximal to the patient's leg or arm or other body part of the patient needing dilation. The physician inserts the distal end 701b of the catheter sheath 701 into the blood vessel 704 of the patient. The physician thereafter advances the hub 202 of the insertion assembly 201 and the balloon dilation catheter 501 with the stent 502 and the balloon 703 enclosed in the insertion device protection apparatus 100 towards the hub 702 of the catheter sheath 701 and allows the hub 702 of the catheter sheath 701 to progressively disengage the first elongate protective cover 101 from the second elongate protective cover 102 from the distal end 100b of the insertion device protection apparatus 100 as exemplarily illustrated in FIG. 8B, while the balloon dilation catheter 501 with the stent 502 and the balloon 703 enters the catheter sheath 701 via the hub 702 of the catheter sheath 701 and advances towards the blood vessel 704. When the insertion device protection apparatus 100 enclosing the advancing balloon dilation catheter 501 with the stent 502 and the balloon 703 contacts the hub 702 of the catheter sheath 701, a resultant pressure on the insertion device protection apparatus 100 at the point of contact between the distal end 100b of the insertion device protection apparatus 100 and the hub 702 of the catheter sheath 701, due to the advancement of the balloon dilation catheter 501 with the stent 502 and the balloon 703, disengages the first elongate protective cover 101 from the second elongate protective cover 102 from the distal end 100b of the insertion device protection apparatus 100, allowing the balloon dilation catheter 501 with the stent 502 and the balloon 703 to enter the catheter sheath 701 uninfected and/or undamaged and/or uncontaminated.

The physician thereafter advances the insertion assembly 201 such that the hub 202 of the insertion assembly 201 applies pressure against the proximal end 100a of the insertion device protection apparatus 100 during the insertion of the balloon dilation catheter 501 with the stent 502 and the balloon 703 into the insertion site 301. The pressure applied at the proximal end 100a of the insertion device protection apparatus 100 due to the advancement of the insertion assembly 201 results in progressive disengagement of the first elongate protective cover 101 from the second elongate protective cover 102 from the proximal end 100a of the insertion device protection apparatus 100 as exemplarily illustrated in FIG. 8C. As the two elongate protective covers 101 and 102 of the insertion device protection apparatus 100 are disengaged from the proximal end 100a and the distal end 100b of the insertion device protection apparatus 100, the physician advances the balloon dilation catheter 501 to insert the stent 502 with the balloon 703 into the hub 702 of the catheter sheath 701, and towards the area of the blood vessel 704 requiring treatment.

As exemplarily illustrated in FIG. 8C, the disengaged elongate protective covers 101 and 102 slide over the insertion assembly 201 and the hub 702 of the catheter sheath 701 as the stent 502 with the balloon 703 progresses through the catheter sheath 701 towards the blood vessel 704 due to the advancement of the balloon dilation catheter 501 of the insertion assembly 201. The stent 502 with the balloon 703 free of the first elongate protective cover 101 and the second elongate protective cover 102 of the insertion device protection apparatus 100 progresses along the catheter sheath 701 into the blood vessel 704 through the distal end 701b of the catheter sheath 701. The physician removes the first elongate protective cover 101 and the second elongate protective cover 102 of the insertion device protection apparatus 100 after the stent 502 with the balloon 703 is completely inserted into the insertion site 301. The physician further advances the balloon dilation catheter 501 with the stent 502 and the balloon 703 inside the blood vessel 704 until a desired location is reached. The physician positions the stent 502 inside the blood vessel 704 by inflating the balloon 703 using the balloon dilation catheter 501 as exemplarily illustrated in FIG. 8D. The physician then deflates the balloon 703 using the balloon dilation catheter 501 and removes the balloon 703 from the blood vessel 704 leaving the stent 502 inside the blood vessel 704 of the patient.

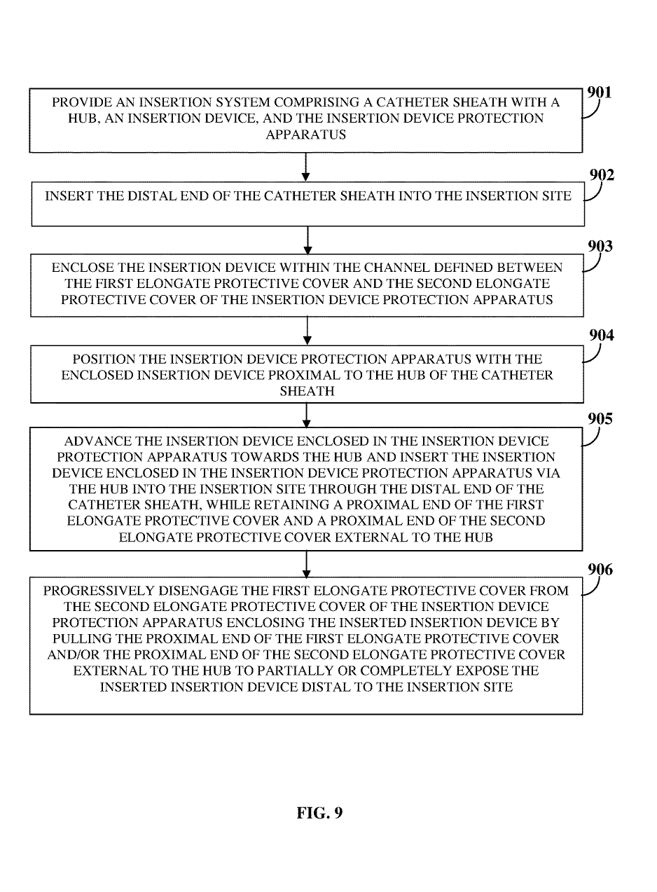

FIG. 9 illustrates an embodiment of the method for securely inserting an insertion device, for example a balloon dilation catheter 501 with a stent 502 and a balloon 703 into an insertion site 301, while protecting the insertion device from infection and/or damage before, during, and after insertion of the insertion device into the insertion site 301. In this embodiment, an insertion system 200 comprising an introducer sheath, for example, a catheter sheath 1001 with a hub 1002 exemplarily illustrated in FIGS. 10A-10D, an insertion device, and the insertion device protection apparatus 100 is provided 901. A proximal end 1001a of the catheter sheath 1001 is connected to the hub 1002. A user, for example, a medical practitioner inserts 902 a distal end 1001b of the catheter sheath 1001 into the insertion site 301 as exemplarily illustrated in FIGS. 10A-10D. The user encloses 903 the insertion device within the channel 105 defined between the first elongate protective cover 101 and the second elongate protective cover 102 of the insertion device protection apparatus 100 exemplarily illustrated in FIG. 1. The tip of the insertion device is enclosed within the defined channel 105. The first elongate protective cover 101 and the second elongate protective cover 102 of the insertion device protection apparatus 100 are positioned to enclose the insertion device. The user positions 904 the insertion device protection apparatus 100 with the enclosed insertion device proximal to the hub 1002 of the catheter sheath 1001.

The user advances 905 the insertion device enclosed in the insertion device protection apparatus 100 towards the hub 1002 of the catheter sheath 1001 and inserts the insertion device enclosed in the insertion device protection apparatus 100 via the hub 1002 into the insertion site 301 through the distal end 1001b of the catheter sheath 1001, while retaining a proximal end 101a of the first elongate protective cover 101 and a proximal end 102a of the second elongate protective cover 102 of the insertion device protection apparatus 100 exemplarily illustrated in FIG. 9A, external to the hub 1002. The user progressively disengages 906 the first elongate protective cover 101 from the second elongate protective cover 102 of the insertion device protection apparatus 100 enclosing the inserted insertion device by pulling the proximal end 101a of the first elongate protective cover 101 and/or the proximal end 102a of the second elongate protective cover 102 external to the hub 1002 to partially or completely expose the inserted insertion device distal to the insertion site 301, for example, at a treatment site 704 such as a blood vessel below the insertion site 301 exemplarily illustrated in FIGS. 10A-10D. For example, the user retracts the first elongate protective cover 101 through the hub 1002 by pulling the first elongate protective cover 101 that is slidably engaged with the second elongate protective cover 102 from the treatment site 704, and then retracts the second elongate protective cover 102 through the hub 1002 by pulling the second elongate protective cover 102 from the treatment site 704.

In an embodiment, the user partially or completely removes the first elongate protective cover 101 from the second elongate protective cover 102 of the insertion device protection apparatus 100 by pulling the proximal end 101a of the first elongate protective cover 101 through the hub 1002 to partially or completely expose the inserted insertion device distal to the insertion site 301. In another embodiment, the user partially or completely removes the second elongate protective cover 102 from the first elongate protective cover 101 of the insertion device protection apparatus 100 by pulling the proximal end 102a of the second elongate protective cover 102 through the hub 1002 to partially or completely expose the inserted insertion device distal to the insertion site 301. In another embodiment, after the first elongate protective cover 101 is removed from the second elongate protective cover 102, the user partially or completely removes the second elongate protective cover 102 from the exposed, inserted insertion device by pulling the proximal end 102a of the second elongate protective cover 102 through the hub 1002 to partially or completely expose the inserted insertion device distal to the insertion site 301. In an embodiment, the first elongate protective cover 101 is completely disengaged from the second elongate protective cover 102. In an embodiment, the insertion device protection apparatus 100 enclosing the insertion device can be removed after insertion into the treatment site 704, but before reaching the treatment site 704. In this embodiment, after the insertion device protection apparatus 100 is removed, the insertion device free of the insertion device protection apparatus 100 is advanced further into the treatment site 704. Since the insertion device enclosed by the insertion device protection apparatus 100 is inserted into the insertion site 301 prior to the progressive disengagement and removal of the first elongate protective cover 101 and the second elongate protective cover 102 from the treatment site 704, the insertion device protection apparatus 100 disclosed herein protects the insertion device before, during, and in this embodiment, after insertion of the insertion device into the insertion site 301.

FIGS. 10A-10D exemplarily illustrate elevation views of the insertion system 200, showing progressive disengagement and removal of two elongate protective covers 101 and 102 of the insertion device protection apparatus 100 to expose an insertion device distal to an insertion site 301, after insertion of the insertion device enclosed in the insertion device protection apparatus 100 into the insertion site 301. The insertion site 301 is, for example, a patient's skin 301 above a treatment site 704 such as a blood vessel on the patient's leg or arm or other body part of the patient needing dilation. The treatment site 704 is herein referred to as the “blood vessel” and referenced by the numeral 704. The insertion device is, for example, a balloon dilation catheter 501 with a balloon 703 positioned within a stent 502 as exemplarily illustrated in FIGS. 10A-10D. In this embodiment, the insertion device protection apparatus 100 protects the stent 502 with the balloon 703 from infection and/or damage and/or contamination internally at the blood vessel 704 during and after the insertion of the stent 502 with the balloon 703 into the insertion site 301, after which the insertion device protection apparatus 100 is removed from the blood vessel 704 having protected the stent 502 with the balloon 703.

A user, for example, a physician, inserts the distal end 1001b of the catheter sheath 1001 into the blood vessel 704 as exemplarily illustrated in FIGS. 10A-10D. The physician inserts the insertion device protection apparatus 100 enclosing the stent 502 with the balloon 703 through the hub 1002 of the catheter sheath 1001 and into the blood vessel 704 through the distal end 1001b of the catheter sheath 1001, while retaining the proximal end 101a of the first elongate protective cover 101 and the proximal end 102a of the second elongate protective cover 102 of the insertion device protection apparatus 100 external to the hub 1002 as exemplarily illustrated in FIG. 10A. The insertion device protection apparatus 100 that encloses the stent 502 with the balloon 703 extending from the balloon dilation catheter 501 protects the balloon dilation catheter 501 with the stent 502 and the balloon 703 from infection and/or damage and/or contamination before, during, and in this embodiment, after the stent 502 with the balloon 703 is inserted into the blood vessel 704.

The physician advances the balloon dilation catheter 501 until the stent 502 with the balloon 703 enclosed by the insertion device protection apparatus 100 reaches a desired location inside the blood vessel 704. The physician then progressively disengages the first elongate protective cover 101 from the second elongate protective cover 102 of the insertion device protection apparatus 100 enclosing the stent 502 with the balloon 703 by pulling the proximal end 101a of the first elongate protective cover 101 through the hub 1002 of the catheter sheath 1001 to partially or completely expose the stent 502 with the balloon 703 as exemplarily illustrated in FIG. 10B. The physician then removes the second elongate protective cover 102 of the insertion device protection apparatus 100 from the partially or completely exposed stent 502 with the balloon 703 by pulling the proximal end 102a of the second elongate protective cover 102 through the hub 1002 of the catheter sheath 1001 to completely expose the stent 502 with the balloon 703 at the blood vessel 704 as exemplarily illustrated in FIG. 10C, after the first elongate protective cover 101 is completely disengaged from the second elongate protective cover 102 of the insertion device protection apparatus 100. The physician then positions the stent 502 inside the blood vessel 704 by inflating the balloon 703 using the balloon dilation catheter 501 as exemplarily illustrated in FIG. 10D. The physician then deflates the balloon 703 using the balloon dilation catheter 501 and removes the balloon 703 from the blood vessel 704 leaving the stent 502 inside the blood vessel 704 of the patient.

The foregoing examples have been provided merely for the purpose of explanation and are in no way to be construed as limiting of the method, the insertion device protection apparatus 100, and the insertion system 200 disclosed herein. The implementations set forth in the foregoing description do not represent all implementations consistent with the subject matter disclosed herein. Instead, they are merely examples consistent with aspects related to the disclosed subject matter and are in no way to be construed as limiting of the method, the insertion device protection apparatus 100, and the insertion system 200 disclosed herein. Wherever possible, the same reference numbers will be used throughout the drawings to refer to the same or like parts.

Although a few variations have been described in detail above, other modifications or additions are possible. In particular, further structures and/or variations can be provided in addition to those set forth herein. For example, the implementations described above can be directed to various combinations and sub-combinations of the disclosed structures and/or combinations and sub-combinations of several further structures disclosed above. In addition, the logic flows and steps for use described herein do not require the particular order shown, or sequential order, to achieve desirable results. Other embodiments can be within the scope of the claims. While the method, the insertion device protection apparatus 100, and the insertion system 200 have been described with reference to various embodiments, it is understood that the words, which have been used herein, are words of description and illustration, rather than words of limitation. Furthermore, although the method, the insertion device protection apparatus 100, and the insertion system 200 have been described herein with reference to particular means, materials, and embodiments, the method, the insertion device protection apparatus 100, and the insertion system 200 are not intended to be limited to the particulars disclosed herein; rather, the method, the insertion device protection apparatus 100, and the insertion system 200 extend to all functionally equivalent structures, methods and uses, such as are within the scope of the appended claims. Those skilled in the art, having the benefit of the teachings of this specification, may effect numerous modifications thereto and changes may be made without departing from the scope and spirit of the method, the insertion device protection apparatus 100, and the insertion system 200 disclosed herein in their aspects.

We claim:

1. An insertion device protection apparatus for protecting an insertion device from infection and/or damage before, and/or during, and/or after insertion of said insertion device into an insertion site, said insertion device protection apparatus comprising:
a first elongate protective cover; and
a second elongate protective cover detachably engaged to said first elongate protective cover, said second elongate protective cover and said first elongate protective cover defining a channel extending through a length of said insertion device protection apparatus for enclosing said insertion device therewithin before, and/or during, and/or after said insertion of said insertion device into said insertion site, wherein said first elongate protective cover is progressively disengaged from said second elongate protective cover from a proximal end and/or a distal end of said insertion device protection apparatus before, and/or during, and/or after said insertion of said insertion device into said insertion site.

2. The insertion device protection apparatus of claim 1, wherein said first elongate protective cover is progressively disengaged from said second elongate protective cover external to said insertion site, while said insertion device is advanced towards said insertion site.

3. The insertion device protection apparatus of claim 1 with said enclosed insertion device configured to be inserted into said insertion site prior to said progressive disengagement and removal of said first elongate protective cover and said second elongate protective cover to expose said insertion device for use.

4. The insertion device protection apparatus of claim 1, wherein said first elongate protective cover comprises at least two grooves, and wherein said second elongate protective cover comprises at least two protrusions for slidably engaging with said at least two grooves of said first elongate protective cover, and wherein said at least two grooves of said first elongate protective cover progressively disengage from said at least two protrusions of said second elongate protective cover for allowing said insertion device to advance down said insertion device protection apparatus during said insertion of said insertion device into said insertion site.

5. The insertion device protection apparatus of claim 1, wherein a tip of said insertion device is enclosed within said channel defined between said first elongate protective cover and said second elongate protective cover.

6. The insertion device protection apparatus of claim 1, wherein said insertion device is selected from the group consisting of a needle, a catheter, a medical balloon, a stent, a balloon catheter, a drug delivery device, a fluid delivery line, and any combination thereof.

7. An insertion system comprising:
an insertion assembly comprising:
a hub; and
an insertion device extending from said hub for insertion into an insertion site; and
an insertion device protection apparatus comprising at least two elongate protective covers positioned to enclose said insertion assembly, said at least two elongate protective covers comprising a first elongate protective cover and a second elongate protective cover detachably engaged to each other, said first elongate protective cover and said second elongate protective cover defining a channel extending through a length of said insertion device protection apparatus for enclosing said insertion device therewithin before, and/or during, and/or after said insertion of said insertion device into said insertion site, wherein said first elongate protective cover is progressively disengaged from said second elongate protective cover from a proximal end and/or a distal end of said insertion device protection apparatus before, and/or during, and/or after said insertion of said insertion device into said insertion site.

8. The insertion system of claim 7, wherein said first elongate protective cover of said insertion device protection apparatus is progressively disengaged from said second elongate protective cover of said insertion device protection apparatus external to said insertion site, while said insertion device is advanced towards said insertion site.

9. The insertion system of claim 7, wherein said insertion device protection apparatus with said enclosed insertion device is configured to be inserted into said insertion site prior to said progressive disengagement and removal of said first elongate protective cover and said second elongate protective cover of said insertion device protection apparatus to expose said insertion device for use.

10. The insertion system of claim 7, wherein said first elongate protective cover of said insertion device protection apparatus comprises at least two grooves, and wherein said second elongate protective cover of said insertion device protection apparatus comprises at least two protrusions for slidably engaging with said at least two grooves of said first elongate protective cover, and wherein said at least two grooves of said first elongate protective cover progressively disengage from said at least two protrusions of said second elongate protective cover for allowing said insertion device to advance down said insertion device protection apparatus during said insertion of said insertion device into said insertion site.

11. The insertion system of claim 7, wherein said insertion assembly further comprises a winged element fixedly attached to said hub and positioned proximal to said insertion device for positioning and affixing said insertion device into said insertion site.

12. The insertion system of claim 7, wherein an effective diameter of said channel defined between said at least two elongate protective covers of said insertion device protection apparatus is less than a diameter of said hub of said insertion assembly.

13. The insertion system of claim 7, wherein a tip of said insertion device is enclosed within said channel defined between said at least two elongate protective covers of said insertion device protection apparatus.

14. The insertion system of claim 7, wherein said insertion device is selected from the group consisting of a needle, a catheter, a medical balloon, a stent, a balloon catheter, a drug delivery device, a fluid delivery line, and any combination thereof.

15. A method for securely inserting an insertion device into an insertion site, while protecting said insertion device from infection and/or damage before and during said insertion of said insertion device into said insertion site, said method comprising:
providing an insertion system comprising:
an insertion assembly comprising:
a hub; and
said insertion device extending from said hub; and
an insertion device protection apparatus comprising at least two elongate protective covers positioned to enclose said insertion assembly, said at least two elongate protective covers comprising a first elongate protective cover and a second elongate protective cover detachably engaged to each other, said first elongate protective cover and said second elongate protective cover defining a channel extending through a length of said insertion device protection apparatus for enclosing said insertion device therewithin before and during said insertion of said insertion device into said insertion site;
positioning said hub and said insertion device of said insertion assembly within said channel defined between said first elongate protective cover and said second elongate protective cover of said insertion device protection apparatus, wherein a tip of said insertion device is enclosed within said defined channel;
positioning said insertion device protection apparatus with said positioned hub and said positioned insertion device of said insertion assembly proximal to said insertion site;
advancing said insertion assembly enclosed in said insertion device protection apparatus towards said insertion site, while said hub of said advancing insertion assembly progressively disengages said first elongate protective cover from said second elongate protective cover from a proximal end of said insertion device protection apparatus; and
inserting said insertion device of said advanced insertion assembly into said insertion site.

16. The method of claim 15, further comprising completely disengaging and removing said first elongate protective cover from said second elongate protective cover of said insertion device protection apparatus that encloses said advanced insertion assembly, after said insertion device of said advanced insertion assembly is completely inserted into said insertion site.

17. The method of claim 15, wherein said insertion device is selected from the group consisting of a needle, a catheter, a medical balloon, a stent, a balloon catheter, a drug delivery device, a fluid delivery line, and any combination thereof.

18. A method for securely inserting an insertion device into an insertion site, while protecting said insertion device from infection and/or damage before and during said insertion of said insertion device into said insertion site, said method comprising:
providing an insertion system comprising:
an introducer sheath, said introducer sheath comprising a proximal end connected to a hub and a distal end that is inserted into said insertion site;
said insertion device; and
an insertion device protection apparatus comprising at least two elongate protective covers positioned to enclose said insertion device, said at least two elongate protective covers comprising a first elongate protective cover and a second elongate protective cover detachably engaged to each other, said first elongate protective cover and said second elongate protective cover defining a channel extending through a length of said insertion device protection apparatus for enclosing said insertion device therewithin before and during said insertion of said insertion device into said insertion site;
inserting said distal end of said introducer sheath into said insertion site;
enclosing said insertion device within said channel defined between said first elongate protective cover and said second elongate protective cover of said insertion device protection apparatus;
positioning said insertion device protection apparatus with said enclosed insertion device proximal to said hub of said introducer sheath;
advancing said insertion device enclosed in said insertion device protection apparatus towards said hub and allowing said hub to progressively disengage said first elongate protective cover from said second elongate protective cover from a distal end of said insertion device protection apparatus, while said insertion device enters said introducer sheath via said hub and advances towards a treatment site distal to said insertion site; and
inserting said insertion device free of said first elongate protective cover and said second elongate protective cover of said insertion device protection apparatus into said insertion site through said distal end of said introducer sheath.

19. The method of claim 18, further comprising completely disengaging and removing said first elongate protective cover from said second elongate protective cover of said insertion device protection apparatus, after said insertion device is completely inserted into said insertion site.

20. The method of claim 18, wherein said insertion device is selected from the group consisting of a needle, a catheter, a medical balloon, a stent, a balloon catheter, a drug delivery device, a fluid delivery line, and any combination thereof.

21. A method for securely inserting an insertion device into an insertion site, while protecting said insertion device from infection and/or damage before, during, and after said insertion of said insertion device into said insertion site, said method comprising:
providing an insertion system comprising:
an introducer sheath, said introducer sheath comprising a proximal end connected to a hub and a distal end that is inserted into said insertion site;
said insertion device; and
an insertion device protection apparatus comprising at least two elongate protective covers positioned to enclose said insertion device, said at least two elongate protective covers comprising a first elongate protective cover and a second elongate protective cover detachably engaged to each other, said first elongate protective cover and said second elongate protective cover defining a channel extending through a length of said insertion device protection apparatus for enclosing said insertion device therewithin before, during, and after said insertion of said insertion device into said insertion site;
inserting said distal end of said introducer sheath into said insertion site;
enclosing said insertion device within said channel defined between said first elongate protective cover and said second elongate protective cover of said insertion device protection apparatus;
positioning said insertion device protection apparatus with said enclosed insertion device proximal to said hub of said introducer sheath;
advancing said insertion device enclosed in said insertion device protection apparatus towards said hub and inserting said insertion device enclosed in said insertion device protection apparatus via said hub into said insertion site through said distal end of said introducer sheath, while retaining a proximal end of said first elongate protective cover and a proximal end of said second elongate protective cover of said insertion device protection apparatus external to said hub; and
progressively disengaging said first elongate protective cover from said second elongate protective cover of said insertion device protection apparatus enclosing said inserted insertion device by pulling said proximal end of said first elongate protective cover and/or said proximal end of said second elongate protective cover external to said hub to expose said inserted insertion device distal to said insertion site.

22. The method of claim 21, further comprising one of partially and completely removing said first elongate protective cover from said second elongate protective cover of said insertion device protection apparatus by pulling said proximal end of said first elongate protective cover through said hub to one of partially and completely expose said inserted insertion device distal to said insertion site.

23. The method of claim 21, further comprising one of partially and completely removing said second elongate protective cover from said first elongate protective cover of said insertion device protection apparatus by pulling said proximal end of said second elongate protective cover through said hub to one of partially and completely expose said inserted insertion device distal to said insertion site.

24. The method of claim 21, wherein said first elongate protective cover is completely disengaged from said second elongate protective cover of said insertion device protection apparatus.

25. The method of claim 21, wherein said insertion device is selected from the group consisting of a needle, a catheter, a medical balloon, a stent, a balloon catheter, a drug delivery device, a fluid delivery line, and any combination thereof.
